(19) United States
(12) Patent Application Publication
(10) Pub. No.: US 2012/0164085 A1
(43) Pub. Date: Jun. 28, 2012

(54) Flavour Compositions

(76) Inventors: John Martin Behan, Kent (GB); David Jonathan Bradshaw, Kent (GB); Jonathan Richards, Kent (GB); Michael John Munroe, Kent (GB); Tony Minhas, Kent (GB); Paula Maria Cawkill, Kent (GB)

(21) Appl. No.: 13/245,559

(22) Filed: Sep. 26, 2011

Related U.S. Application Data
(63) Continuation of application No. 10/545,909, filed on Aug. 17, 2005, now abandoned.

(30) Foreign Application Priority Data
Feb. 18, 2003 (GB) 0303675.3

(51) Int. Cl. A61K 8/97; A61L 101/56; A61L 101/32; A61K 8/18

(52) U.S. Cl. 424/58; 424/49; 424/76.1

(57) ABSTRACT

A flavor composition which includes as, at least 8% of its total weight, (a) at least 0.5% by weight of a peppermint oil containing 1-isopropylidene-4-methyl-2-cyclohexanone in an amount from 1% to 4% by weight, 5-methyl-2-(1-methylethyl)-1-cyclohexanone in an amount from 8% to 13% by weight and less than 0.5% by weight of eucalyptol; and a spearmint oil containing less than 70% by weight of carvone and at least 14% by weight of limonene; or mixtures thereof; and (b) at least 0.5% by weight of two or more of the following: decanol, octanal, allyl hexanoate, anethole, anised rectified, basil oil, benzyl butyrate, camomile oil, cinnamic aldehyde, cis-3-hexenyl acetate, citral natural, citronella ceylon, ethyl heptanoate, eugenol, fennel sweet, geranyl acetate, ionone alpha, lime, orange flavour, para cresyl methyl ether and pinene alpha. The compositions are useful in reducing oral malodors.

### FIELD OF THE INVENTION

This invention relates to flavour compositions, to products containing such flavour compositions, and to the use of a flavour material or flavour composition to deliver a beneficial effect on oral malodour. In particular, the invention relates to flavour materials, and flavour compositions, for reducing or preventing oral malodour.

### BACKGROUND TO THE INVENTION

Oral malodour is caused by bacteria and bacterial activity within the oral cavity. The major components of oral malodour are volatile sulphur compounds (referred to hereinafter for the purposes of brevity and simplicity as “VSCs” or “VSC”), particularly hydrogen sulphide (H₂S) and methyl sulphides such as methyl mercaptan (CH₃SH). These odorous compounds result from the bacterial degradation of exogenous and endogenous amino acids derived from proteinaceous materials e.g. food debris, saliva, gingival crevicular fluid, exfoliated oral epithelia salivary corpuscles or blood, present in the oral cavity. In this process, bacteria firstly hydrolyse proteins (from the proteinaceous material) to their constitutive amino acids. Thiol group-containing amino acids, e.g. cysteine, cystine and methionine, are then broken down to produce VSCs.

Although more than 300 species of bacteria have been isolated from the mouth, the production of VSCs such as hydrogen sulphide and methyl mercaptan has been linked to specific groups of bacteria, particularly gram-negative species. For example, the gram-negative micro-organism Fusobacterium nucleatum is strongly implicated in VSC production (McNamara T F, Alexander J F, Lee M. (1972) The role of micro-organisms in the production of oral malodour. Oral Surg. Oral Med. Oral Pathol.; 34(1): 41-8; Persson S, Edlund M B, Claesson R, Carlsson J. (1990) The formation of hydrogen sulfide and methyl mercaptan by oral bacteria. Oral Microbiol. Immunol. 5(4): 195-201; Solis-Gaffar M C, Fischer T T and Gaffar A. (1979) Instrumental evaluation of odor produced by specific oral micro-organisms. J Soc. Cosmet. Chem. 30: 241-7).

It is known from the scientific literature that micro-organisms responsible for producing VSCs are found in the gingival crevice, the tongue coating and other parts of the oral cavity, with the largest proportion of oral malodour believed to originate from the tongue dorsum area.

A number of approaches are used to combat oral malodour.

A simple approach is to mechanically scrape the surface of the tongue to remove proteinaceous waste materials that are typically degraded to form VSCs. However, such materials form a strong attachment to the oral mucosa and this may result in damage to the underlying tissue if the surface of the tongue is scraped too vigorously.

A further approach is to simply mask oral malodour with materials known to have this effect. The process of odour masking involves using a material which has an agreeable odour in such concentrations that the odour is no longer noticeable. In most cases this approach provides only temporary relief, particularly for oral malodour, since only small amounts of masking odorants (generally minty flavours) may be applied to the oral cavity from a product, so their performance is short-lived.

Various antimicrobial agents may be used in products intended for use in the oral cavity to reduce oral malodour. Antimicrobial agents used in oral care products are designed to reduce the population, inhibit growth or diminish the metabolic activities of micro-organisms present in the oral cavity. Typical agents of this nature include triclosan (2′,4,4′-trichloro-2-hydroxydiphenyl ether), chloride dioxide, chlorhexidine and metronidazole. A number of essential oils, e.g. citral, are also known to exhibit an antimicrobial effect against certain bacteria. The use of such agents in appropriate concentration in an oral care product results in a non-selective antimicrobial action exerted upon most of the oral cavity's natural microflora. That is, antimicrobial agents may indiscriminately target and affect all populations of micro-organisms resident in the oral cavity, including natural microflora. This is an undesirable disadvantage, since the natural microflora provides a protective barrier (colonisation resistance) against, invasion by potentially pathogenic bacteria.

US 2002/0064505 concerns an anti-odour composition comprising a higher alcohol and a taste-masking additive.

WO 98/44901 concerns oral hygiene compositions including an antimicrobial agent selected from cedarwood oil, chloramphenicol, citronella oil, Glycyrrhiza glabra extract, juicy fruit basil oil, lemon basil oil, and Rosmarinus officinalis oil.

U.S. Pat. No. 5,472,684 concerns compositions comprising a combination of thymol and eugenol or a combination of thymol, eugenol and a sesquiterpene alcohol in an oral product. Flavouring agents including Australian Tea Tree oil, chamomile tincture and eucalyptol, can also be added to improve taste.

U.S. Pat. No. 6,197,288 concerns a malodour counteract composition comprising an organoleptically effective amount of one or more specified malodour counteractant agents in an oral care vehicle.

U.S. Pat. No. 5,711,937 concerns an antibody-containing oral composition comprising a flavour component selected from carvone, anethole, cineole, methyl salicylate, eugenol, ethyl butyrate and cinnamic aldehyde; and 1-menthol; where the flavour component and 1-menthol are blended in a weight ratio of from 1:9 to 8:2.

In contrast to the generally broad spectrum antimicrobial approaches for reducing or preventing oral malodour disclosed in the prior art, the present invention is based on the selective inhibition of VSC producing micro-organisms by a flavour material or mixtures thereof.

### SUMMARY OF THE INVENTION

The present invention is thus based on extensive testing of flavour materials to determine whether a particular material is capable of inhibiting the production of odoriferous VSCs by micro-organisms present in the oral cavity. Based on this testing, flavour materials have been identified, which whilst known, may possess hitherto unappreciated oral malodour reducing properties. The invention thus enables flavour compositions to be defined that reduce or prevent oral malodour. Additionally, in a preferred embodiment, the invention enables flavour compositions to be formulated comprising flavour material(s) which selectively target and inactivate the bacteria producing odoriferous VSCs whilst preserving the remaining protective oral cavity microflora.

Accordingly, in one aspect, the present invention provides a flavour composition which is a mixture of flavour materials, characterised in that the flavour composition comprises at least 8% by weight of the total weight of the flavour composition of ingredients selected from the following groups of flavour materials:

(a) at least 0.5% by weight of the flavour composition of one or more of the following: a peppermint oil comprising 1-isopropylidene-4-methyl-2-cyclohexanone in an amount from 1% to 4% by weight, 5-methyl-2-(1-methylethyl)-1-cyclohexanone in an amount from 8% to 13% by weight and less than 0.5% by weight of eucalyptol; a spearmint oil comprising less than 70% by weight of carvone and at least 14% by weight of limonene; or mixtures thereof; and
(b) at least 0.5% by weight of the flavour composition of two or more of the following: decanol, octanal, allyl hexanoate, anethole, aniseed rectified, basil oil, benzyl butyrate, camomile oil, cinnamic aldehyde, cis-3-hexenyl acetate, citral natural, citronella ceylon, ethyl heptanoate, eugenol, fennel sweet, geranyl acetate, ionone alpha, lime, orange flavour, para cresyl methyl ether, pinene alpha.

The ingredients of the composition are known flavour materials which are readily available commercially in grades suitable for various intended purposes. Details of the flavour materials and potential suppliers thereof are mentioned, for example, in “Allured's Flavor and Fragrance Materials 2002”, Allured Publishing Corp., Carol Stream, Ill., USA, ISBN 0-931710-84-7.

Preferably, group (a) and group (b) flavour materials together comprise at least 20% by weight of the total weight of the flavour composition and more preferably at least 40% by weight.

The peppermint oil and/or spearmint oil is typically of natural or synthetic origin, preferably of natural origin.

The components of a particular peppermint oil or spearmint oil and the relative amounts of each component can be readily determined by a person skilled in the art, e.g. using known analytical techniques.

Examples of peppermint oils suitable for use herein include Peppermint Indian Rectified (all grades), Peppermint American Far West Bulked, Peppermint American Willamette Natural.

Preferably, the peppermint oil comprises 1-isopropylidene-4-methyl-2-cyclohexanone (pulegone) in an amount from 1.5% to 3% by weight, 5-methyl-2-(1-methylethyl)-1-cyclohexanone (iso-menthone) in an amount from 8.5% to 12.5% by weight and less than 0.5% by weight of eucalyptol.

One or more peppermint oils which include 1-isopropylidene-4-methyl-2-cyclohexanone, 5-methyl-2-(1-methylethyl)-1-cyclohexanone and eucalyptol in the specified amounts may be present in a flavour composition. Such peppermint oils may also be mixed with other peppermint oils which do not fulfil the requirements for a peppermint oil useful herein.

Examples of suitable spearmint oils for use herein include Spearmint American Far West Scotch, Spearmint Bulked Extra and Spearmint American Far West Native Redistilled.

One or more spearmint oils which include less than 70% by weight of carvone and at least 14% by weight of limonene may be present in a flavour composition. Other spearmint oils which do not meet these requirements may also be present.

Group (b) flavour materials useful herein include:

Decanol (alcohol C10);
Octanal (aldehyde C8);
Allyl hexanoate;
Anethole (p-methoxypropenyl benzene);

### Aniseed Rectified;

Basil oil which is conveniently basil comores;
Benzyl butyrate;
Camomile oil which is conveniently camomile English Distilled;
Cinnamic aldehyde which is conveniently cinnamic aldehyde extra, available from Quest International;
Cis-3-hexenyl acetate;

### Citral Natural;

### Citronella Ceylon;

Ethyl heptanoate;

### Eugenol;

### Fennel Sweet;

Geranyl acetate;
Ionone alpha (4-(2,6,6-trimethylcyclohex-2-ene-1-yl)but-3-ene-2-one) (available from Quest International);

### Lime;

Orange Flavour, particularly Orange Flavour Artificial which is the trade name of an orange material available from Givaudan;
Para cresyl methyl ether;
Pinene alpha.

A flavour composition in accordance with the invention preferably comprises at least 3 and more preferably at least 5 flavour materials from group (b).

Flavour compositions of the invention preferably comprise at least 5% by weight, and more preferably at least 15% by weight, of the flavour composition of group (a) flavour materials.

Flavour compositions of the invention preferably comprise at least 7% by weight, more preferably at least 10% by weight, even more preferably at least 20% by weight and most preferably at least 30% by weight, of the flavour composition of group (b) flavour materials.

Also included within the scope of the invention is a method, particularly a cosmetic method, for reducing or preventing oral malodour by introducing in the oral cavity a flavour composition which is a mixture of flavour materials, characterised in that the flavour composition comprises at least 8% by weight of the total weight of the flavour composition of ingredients selected from the following groups of flavour materials:

(a) at least 0.5% by weight of the flavour composition of one or more of the following: a peppermint oil comprising 1-isopropylidene-4-methyl-2-cyclohexanone in an amount from 1% to 4% by weight, 5-methyl-2-(1-methylethyl)-1-cyclohexanone in an amount from 8% to 13% by weight and less than 0.5% by weight of eucalyptol; a spearmint oil comprising less than 70% by weight of carvone and at least 14% by weight of limonene; or mixtures thereof; and
(b) at least 0.5% by weight of the flavour composition of two or more of the following: decanol, octanal, allyl hexanoate, anethole, aniseed rectified, basil oil, benzyl butyrate, camomile oil, cinnamic aldehyde, cis-3-hexenyl acetate, citral natural, citronella ceylon, ethyl heptanoate, eugenol, fennel sweet, geranyl acetate, ionone alpha, lime, orange flavour, para cresyl methyl ether, pinene alpha.

For example, the efficacy with which a flavour composition of the invention reduces or prevents oral malodour can be determined as described in Example 5 below, e.g. by testing the composition in a Malodour Counteraction Panel Test.

In a further aspect, the invention provides a method, particularly a cosmetic method, for reducing or preventing the production of odoriferous volatile sulphur compounds in the oral cavity, the method comprising the step of introducing in the oral cavity a flavour composition in accordance with the invention.

For example, the efficacy with which a particular flavour composition in accordance with the invention reduces or prevents the production of odoriferous VSCs may be measured as described in Example 6 below, using a Halimeter device (where Halimeter is a Trade Mark).

In an even further aspect, the invention provides a method for inhibiting the bacterial production in vitro of odoriferous volatile sulphur compounds, by introducing a flavour composition in accordance with the invention to a bacterial culture.

For example, the inhibition of production of odoriferous VSCs in vitro by bacteria, e.g. Klebsiella pneumoniae and Fusobacterium nucleatum, by a flavour material useful in a composition of the invention, or by compositions of the invention as such, may be measured as described in Examples 1(a) and 1(b) below. Typically, the bacteria are inhibited at a concentration below the MIC value of the flavour material/flavour composition for the bacteria.

The flavour materials useful in a flavour composition of the invention are capable of reducing or preventing oral malodour by inhibiting the production of odoriferous VSCs by micro-organisms present in the oral cavity. In particular, the flavour materials are capable of inhibiting the production of hydrogen sulphide. Typically, the specified flavour materials inhibit the production of odoriferous VSCs, particularly hydrogen sulphide, by the gram-negative bacteria Klebsiella pneumoniae and Fusobacterium nucleatum present in the oral cavity.

One property that characterises the effectiveness of a compound, e.g. a flavour material, to inhibit the production of VSCs produced by particular micro-organisms in the oral cavity, is the minimum inhibitory concentration, or MIC, of the compound. The MIC is the minimum amount of a compound (e.g. in ppm) at which no bacterial growth is observed. Generally, the lower the MIC of a compound for a bacterium, the more effective the compound will be at inhibiting bacterial growth. At concentrations above the MIC, a compound may act by directly killing existing viable bacteria or inhibiting the growth and reproduction of the bacteria (antimicrobial effect). At concentrations below the MIC, a compound may interfere with the metabolic process, e.g. by inactivating bacteria producing malodorous compounds, but typically does not inhibiting the growth and reproduction of bacteria (sub-lethal or sub-MIC effect).

The inhibitory effect of a flavour composition comprising the flavour materials useful herein can be achieved antimicrobially, or more surprisingly, sub-lethally.

The antimicrobial effects of compounds, e.g. flavour materials, are usually divided into two types; they can either inhibit bacterial growth (bacteriostatic action) or alternatively they can act by directly killing existing viable bacteria (bactericidal action).

The bacteriostatic action of a compound “X” (such as a flavour material) against a particular bacterium, can be tested for in vitro by inoculating a standard, small number of bacteria into broths containing an appropriate range of concentrations of X. The broths are then incubated for a suitable time, and growth compared with a control containing no inhibitor. The broth containing the lowest concentration of X which shows reduction of growth compared to the control broth, is defined as the minimum inhibitory concentration (MIC).

The determination of the bactericidal action of a compound “Y” (such as a flavour material) is carried out by adding various concentrations of compound Y to replicate broths containing relatively high, standard numbers of bacteria. After a certain period allowing any antibacterial activity to take place, aliquots of the bacterial cultures are diluted (usually in 10-fold steps) and dispensed onto agar plates. The plates are incubated with the expectation that each viable cell should produce a visible colony. The numbers of colonies are multiplied to take account of the dilution, to establish the number of viable cells in the broths. Once again, the broths containing compound Y are compared with an untreated control broth. The minimum concentration of compound Y which causes a reduction in the viable number of bacteria is the minimum bactericidal concentration (MBC). MBC can also be expressed in terms of the MBC required to produce a certain degree of killing (for example, a 3 log₁₀ reduction in count, equivalent to a 99.9% kill). Still further, the MBC can be expressed in kinetic terms—the time of exposure to an agent required for a given MBC effect.

A further possibility is that the process of inhibition could be sub-lethal (or sub-MIC), whereby the flavour materials interfere with the metabolic process, but typically do not inhibit bacterial growth.

Typically, the bacterial production of VSCs is reduced by at least 40%. For example, for group (a) and group (b) flavour materials a VSC reduction value, measured as described in Examples 1(a) or 1(b), of at least 40% is obtained at a concentration of 500 ppm of a flavour material. The group (a) and group (b) flavour materials surprisingly demonstrate good activity against the specific bacteria. For example, the MIC value for each flavour material of group (b) for the bacteria Klebsiella pneumoniae and Fusobacterium nucleatum is typically greater than 1000 ppm (0.1%), whilst the peppermint and spearmint oils of group (a) typically have MIC values of 2500 ppm (0.25%) or about 5000 ppm (0.5%) for the bacteria Klebsiella pneumoniae. Materials having such MIC values would typically be considered to be ineffective at inhibiting the VSC producing micro-organisms and hence have poor oral malodour activity. As described herein above, typically, the lower the MIC value of a material, the more effective the material is at inhibiting bacterial growth.

Three modes of achieving a reduction in odoriferous VSC production are possible. In the first mode, the flavour materials (or flavour compositions) may act by direct (overt antimicrobial) killing of oral cavity bacteria, e.g. by more than 10-fold; in the second mode, they may act on odoriferous VSC generation whilst maintaining a microbial cell viability of at least 70%; in the third mode, they may inhibit odoriferous VSC generation, at a concentration below the minimum inhibitory concentration (MIC), determined as described in Example 2 below. The third mode is preferred, since this provides oral malodour counteraction benefits, whilst leaving the natural oral cavity microflora undisturbed. Thus, preferably, the bacterial production of odoriferous VSCs can be reduced or eliminated without significantly disturbing the oral cavity's natural microflora. This may be achieved by inhibiting the bacteria responsible for the production of odoriferous VSCs, in particular Klebsiella pneumoniae and Fusobacterium nucleatum, at a concentration below the MIC.

For antimicrobial action, it may be useful for the composition to include one or more of the following materials: tea tree oil, aldehyde C9 (nonanal), Orange Oil Terpeneless and aldehyde C10 (decanal). Citraldone (Citraldone is a Trade Mark and is available from Bush Boake Allen as Citraldone NA4065) may also be useful. Such materials may be present in an amount in the range 0.01% to 1.0% by weight or more of the total weight of the flavour composition, and typically demonstrate a VSC reduction value of at least 40% at a concentration of 500 ppm as measured by the method described in Example 1(a) or 1(b), whilst having a corresponding MIC of less than 1000 ppm.

In an even further aspect the present invention provides use of one or more of the following flavour materials: octanal, allyl hexanoate, anethole, aniseed rectified, basil oil, benzyl butyrate, camomile oil, cinnamic aldehyde, cis-3-hexenyl acetate, ethyl heptanoate, fennel sweet, ionone alpha, lime, orange flavour, para cresyl methyl ether, pinene alpha, a spearmint oil comprising less than 70% by weight of carvone and at least 14% by weight of limonene, a peppermint oil comprising 1-isopropylidene-4-methyl-2-cyclohexanone in an amount from 1% to 4% by weight, 5-methyl-2-(1-methylethyl)-1-cyclohexanone in an amount from 8% to 13% by weight and less than 0.5% by weight of eucalyptol; for the purpose of reducing and/or preventing oral malodour.

The flavour composition typically also includes other flavour ingredients (which may be selected from the 400-500 or so flavour materials that are in current use when formulating flavour compositions) chosen to give desired overall flavour characteristics to the composition.

The flavour composition of the invention can be readily made by simply mixing the specified ingredients, as is well known to those skilled in the art.

The flavour compositions of the invention find application in a wide range of consumer products, particularly oral care products such as toothpastes, mouthwashes, chewing gum (where the term “chewing gum” is intended also to encompass bubble gum), dental floss, dissolvable mouth films, breath sprays and breath freshening tablets.

The present invention also includes within its scope consumer products, particularly oral care products, including a flavour composition in accordance with the invention.

The consumer products, particularly oral care products, which include a flavour composition in accordance with the invention may be formulated in a conventional manner as is well known to those skilled in the art. For example, a toothpaste formulation will typically include from 0.3% to 2.0% by weight, preferably from 0.5% to 1.5% by weight and more preferably from 0.8% to 1.2% by weight, of the flavour composition. A mouthwash will typically contain the flavour composition in an amount in the range 0.05% to 2.0% by weight, preferably 0.1% to 1.0% by weight, and more preferably 0.15% to 0.5% by weight. For a chewing gum, the composition of the invention may be present in an amount in the range 0.5% to 3.5% by weight, preferably 0.75% to 2.0% by weight and more preferably 1.0% to 1.75% by weight.

A consumer product may conveniently also include ingredients such as salts of zinc, triclosan, salts of copper, strontium, tin (stannous), peroxides, chlorite, pyrophosphates, sodium dodecyl sulphate/sodium lauryl sulphate (SDS/SLS), fluoride, parabens, cetylpyridinium chloride, sanguinarine, or chlorhexidine; to help deliver oral malodour counteraction benefits in-use.

The invention will be illustrated by the following examples and with reference to the accompanying drawings in which:

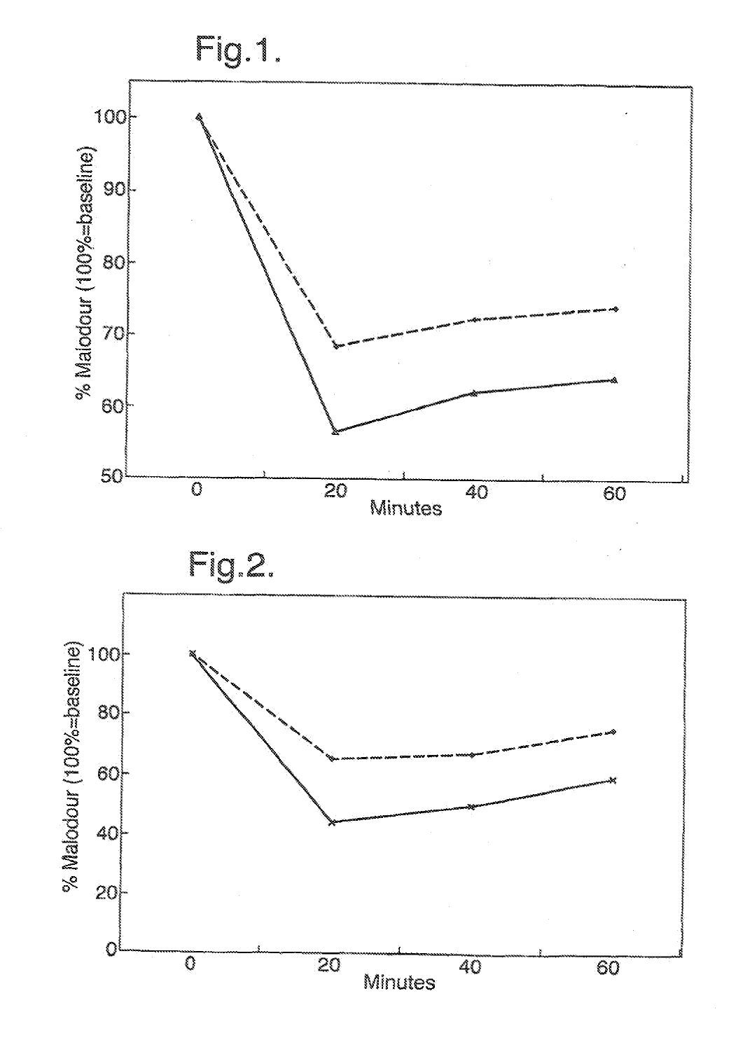

FIG. 1 is a graph of malodour score (percent) versus time (minutes) for a toothpaste containing flavour composition E (see below) (represented by a full line with filled in triangles) and an unflavoured toothpaste (represented by a dashed line with filled in diamonds), showing results of breath malodour scores before (t=0) and after brushing with each toothpaste, where a malodour score is compared against a baseline set to one hundred percent at t=0; and

FIG. 2 is a graph similar to FIG. 1, but showing results for a toothpaste containing flavour composition F (see below) (represented by a full line with crosses) and an unflavoured toothpaste (represented by a dashed line with filled in diamonds).

The methods described in Examples 1, 5 and 6 below are simply indicative of the performance of a flavour composition in accordance with the invention (or flavour material useful therein). For a flavour composition to be commercially useful, it is not necessary for the composition to perform well in all of the described methods, i.e. a flavour composition that performs well in one method, but not another is not necessarily a poor composition.

### EXAMPLE 1

### VSC Reduction Assays

### EXAMPLE 1(a)

### Aerobic VSC Reduction Assay

The ability of a flavour material (or flavour composition) to inhibit the production of hydrogen sulphide under aerobic conditions was determined using the following method.

A bacterial culture of the micro-organism Klebsiella pneumoniae ATCC 10031 (American Type Culture Collection (ATCC), P.O. Box 1549, Manassas, Va. 20108, USA) was grown overnight at 37° C. in Tryptone Soya Broth (TSB) (Oxoid, Basingstoke, UK). The bacterial culture was harvested by centrifugation at 3555 g for 10 minutes. The cells obtained were then washed three times with sterile 0.3% TSB and resuspended in 12 ml of 0.3% TSB. The optical density of the suspension was measured using a Pye Unicam 8620 spectrophotometer (Pye Unicam, Cambridge, UK) and then adjusted by the addition of 0.3% TSB to give an optical density of 39 at a wavelength of 610 nm. Sterile cysteine was then added to give a final concentration of 5.0% w/v in the reaction vessel. The mixture was then incubated at 37° C., with shaking at 200 rpm for 3 hours to induce cysteine metabolism in the micro-organisms. After this time, 1 ml aliquots of the bacterial suspension were dispensed into vials containing 11.55 ml of 0.3% TSB, then 200 μl of 32,000 ppm stock solution of flavour material or flavour composition was added to give a final concentration in the vials of 500 ppm. 250 μl of a 2% w/v cysteine solution was then added to each vial, and the vials capped to produce an air-tight seal. The vials were then incubated for a further hour at 37° C., with shaking at 200 rpm. After this time, 500 μl of the headspace gas was removed and the quantity of hydrogen sulphide (H₂S) present in the sample estimated using GC analysis.

GC conditions for analysis:—

### GC: Carlo Erba Instruments, GC Mega2 Series

Column: CP-SIL 8 column
25 m×0.32 mm (internal diameter)×0.4 μm (film thickness)

### Detector: SSD 250

### Oven Temperature: 70° C.

### Injection Temperature: 150° C.

### Split Ratio: 50:1

### EXAMPLE 1(b)

### Anaerobic VSC Reduction Assay

The ability of a flavour material (or flavour composition) to inhibit the production of hydrogen sulphide under anaerobic conditions was determined using the following method.

A culture of the micro-organism Fusobacterium nucleatum ATCC 10953 (American Type Culture Collection (ATCC), P.O. Box 1549, Manassas, Va. 20108, USA) was inoculated into 250 ml of pre-reduced Schaedler Anaerobic Broth (SAB) (Oxoid, Basingstoke, UK) and incubated anaerobically for 48 hours.

In an anaerobic cabinet (Compact Anaerobic Workstation, Don Whitley Scientific, Shipley, West Yorkshire) headspace vials were inoculated with 9 ml of the Fusobacterium nucleatum culture (with an approximate optical density of the culture at 610 nm of 0.5 (OD₆₁₀=0.5)). To each vial was added 1.0 ml of 2% w/v cysteine solution to bring the final volume in the vials to 10 ml. A stock solution of concentration 32,600 ppm of flavour material/flavour composition was prepared by adding 5 ml of 0.3% SAB to 163 mg of flavour material/flavour composition. Flavour material/flavour composition was then added to the vials (except control) to give a final concentration of 500 ppm. Sterile vial caps were sealed onto the vials and these vials then incubated at 37° C. with shaking at 160 rpm for four hours. After the incubation period, the hydrogen sulphide content of the headspace gas in the vials was analysed by GC using the conditions indicated above.

Experiments demonstrated that the results of the assays described in Examples 1(a) and 1(b), used to measure the inhibition of VSC production, particularly hydrogen sulphide, by a flavour material (or flavour composition), were comparable for more than 95% of materials tested.

### EXAMPLE 2

### Determination of Minimum Inhibitory Concentration (MIC) of Flavour Materials or Flavour Compositions

The MIC of a flavour material or flavour composition was determined by the following method.

A fresh culture of the test inoculum Klebsiella pneumoniae (as above) was diluted in sterile 0.1% special peptone solution to give a concentration of approximately 10⁶ colony forming units (cfu) per ml.

Test samples of flavour material or flavour composition were diluted in sterile tryptone soya broth (TSB) to give an initial stock solution, typically of concentration 40,000 ppm (4% v/v). However, it will be appreciated that the concentration of the initial stock solution of flavour material/flavour composition can be varied if desired to investigate a different range of concentrations. Each row of a standard, 96-well plastic microtitre plate (labelled A-H) was allocated to one sample, i.e. eight samples per plate. Row H contained only TSB for use as a bacterial control to indicate the degree of turbidity resulting from bacterial growth in the absence of any test material. Aseptically, 200 μl of the initial dilution of flavour material/flavour composition was transferred to the 1^{st} and 7^{th} well of the appropriate row. All other test wells were filled with 100 μl of sterile TSB using an 8-channel micro-pipette. The contents of each of the wells in column 1 were mixed by sucking samples up and down the pipette tips, before 100 μl was transferred to column 2. The same sterile pipette tips were used to transfer 100 μl of each well in column 7 into the appropriate well in column 8. This set of eight tips was then discarded into disinfectant solution. Using eight fresh, sterile tips the process was repeated by transferring 100 μl from column 2 into column 3 (and into 8 and 9). The process was continued until all the wells in columns 6 and 12 contained 200 μl. After mixing, 100 μl was discarded from wells in columns 6 and 12 to waste. Finally, 100 μl of pre-diluted bacterial test culture (approx 10⁶ cfu/ml) was added, thus giving a final volume of 200 μl in each well.

A blank plate was prepared for each set of eight samples in exactly the same way, except that 100 μl of sterile 0.1% TSB was added instead of the bacterial culture. This plate was used as the control plate against which the test plate(s) could be read.

Test and control plates were then sealed using autoclave tape and incubated at 37° C. for 24 hours. The wells were examined after 24 hours for turbidity to determine if the material had inhibited growth or not.

A microtitre plate reader (Model MRX, Dynatech Laboratories) was present to gently agitate the plates and mix the contents. The absorbance at 540 nm (hereinafter referred to for brevity and simplicity as “A₅₄₀”) was used as a measure of turbidity resulting from bacterial growth. The control, un-inoculated plate for each set of samples was read first, and the plate reader then programmed to use the control readings to blank all other plate readings for the inoculated plates for the same set of test materials (i.e. removing turbidity due to flavour and possible colour changes during incubation). Thus, the corrected readings generated were absorbances resulting from turbidity from bacterial growth. The MIC was taken as the lowest concentration of flavour material/flavour composition required to inhibit growth so that the change in absorbance during the incubation period was <0.2 A₅₄₀.

### EXAMPLE 3

### Flavour Compositions

### Flavour Composition A

A flavour composition in accordance with the invention of a peppermint, spearmint, wintergreen, cinnamon nature was prepared by mixing the following ingredients.

| Ingredient | % w/w |
|---|---|
| Anethole Synthetic* | 9.00 |
| Cinnamic Aldehyde Extra* | 3.50 |
| Eucalyptol | 1.00 |
| Irone Alpha (10% in propylene glycol) | 0.10 |
| Lime* | 1.50 |
| Menthol Laevo Extra | 35.4 |
| Methyl Salicylate | 8.00 |
| Peppermint American Willam Redis | 14.9 |
| Peppermint Indian Rectified** | 20.5 |
| Spearmint American Far West Scotch** | 6.00 |
| Vanillin | 0.10 |
| | 100.00 |

*Group (b) materials
**Group (a) materials

This composition gave a VSC reduction value of 67% when tested using the method described in Example 1(a) above.

### Flavour Composition B

A flavour composition in accordance with the invention of a peppermint, wintergreen nature was prepared by mixing the following ingredients.

| Ingredient | % w/w |
|---|---|
| Allyl Hexanoate* | 0.30 |
| Anethole Synthetic* | 6.50 |
| Bananate | 1.20 |
| Benzyl Butyrate* | 0.20 |
| Clove Bud Rectified Extra | 3.80 |
| Lime* | 0.80 |
| Menthol Laevo Extra | 43.3 |
| Methyl Salicylate | 23.7 |
| Orange Flavour Artificial (GIV)* | 0.10 |
| Orange Florida | 0.50 |
| Orange Terpeneless | 0.10 |
| Peppermint American Far West BLKD** | 0.60 |
| Peppermint American Yakima Rectified | 13.3 |
| Peppermint Moroccan | 4.70 |
| Pinarene | 0.90 |
| | 100.00 |

*Group (b) materials
**Group (a) materials

Bananate and Pinarene are trade names and are available from Quest International.

This composition gave a VSC reduction value of 75% when tested using the method described in Example 1(a) above.

### Flavour Composition C

A flavour composition in accordance with the invention of a peppermint nature was prepared by mixing the following ingredients.

| Ingredient | % w/w |
|---|---|
| Allyl Hexanoate* | 5.00 |
| Anethole Synthetic* | 6.20 |
| Bananate | 24.6 |
| Benzyl Butyrate* | 4.90 |
| Lime* | 15.0 |
| Menthol Laevo Extra | 11.1 |
| Orange Flavour Artificial (GIV)* | 2.00 |
| Orange Terpeneless | 0.80 |
| Peppermint American Far West BLKD** | 12.4 |
| Pinarene | 18.0 |
| | 100.00 |

*Group (b) materials
**Group (a) materials

Bananate and Pinarene are trade names and are available from Quest International.

This composition gave a VSC reduction value of 83% when tested using the method described in Example 1(a) above.

### Flavour Composition D

A flavour composition in accordance with the invention of a peppermint, wintergreen nature was prepared by mixing the following ingredients.

| Ingredient | % w/w |
|---|---|
| Allyl Hexanoate* | 0.05 |
| Anethole Synthetic* | 8.44 |
| Bananate | 0.25 |
| Benzyl Butyrate* | 0.05 |
| Clove Base ABF0917 | 6.25 |
| Lime* | 0.15 |
| Menthol Laevo Extra | 42.11 |
| Methyl Salicylate | 26.48 |
| Orange Flavour Artificial (GIV)* | 0.02 |
| Orange Terpeneless | 0.01 |
| Peppermint American Far West BLKD** | 0.12 |
| Peppermint American Willamette Natural** | 15.89 |
| Pinarene | 0.18 |
| | 100.00 |

*Group (b) materials
**Group (a) materials

Bananate and Pinarene are trade names and are available from Quest International.

This composition gave a VSC reduction value of 82% when tested using the method described in Example 1(a) above.

### Flavour Composition E

A flavour composition in accordance with the invention of a peppermint, wintergreen, spice nature was prepared by mixing the following ingredients.

| Ingredient | % w/w |
|---|---|
| Anethole Synthetic* | 9.0 |
| Cinnamic Aldehyde* | 3.5 |
| Lime Oil* | 1.5 |
| Menthol Laevo | 35.4 |
| Methyl Salicylate | 8.0 |
| Peppermint Oil (within the specified limits)** | 35.4 |
| Pinarene | 0.5 |
| Bananate | 0.2 |
| Spearmint Oil (within the specified limits)** | 6.4 |
| Vanillin | 0.1 |
| | 100.00 |

*Group (b) materials
**Group (a) materials

Pinarene and Bananate are trade names and are available from Quest International.

This composition gave a VSC reduction value of 79% when tested using the method described in Example 1(a) above.

### Flavour Composition F

A flavour composition in accordance with the invention of a spearmint, peppermint nature was prepared by mixing the following ingredients.

| Ingredient | % w/w |
|---|---|
| Anethole Synthetic* | 12.00 |
| Cis-3-Hexenyl Acetate* | 0.20 |
| Lemon Oil | 1.00 |
| Menthol Laevo | 10.72 |
| Peppermint Oil (within the specified limits)** | 56.00 |
| Spearmint Oil (within the specified limits)** | 20.08 |
| | 100.00 |

*Group (b) materials
**Group (a) materials

This composition gave a VSC reduction value of 72% when tested using the method described in Example 1(a) above.

### EXAMPLE 4

### Formulations

Either of flavour compositions C or F above may be included in the following chalk toothpaste which is prepared according to conventional methods known to those skilled in the art:

### Chalk Toothpaste

| Material | % w/w |
|---|---|
| Glycerine | 20.0 |
| Distilled Water | 35.3 |
| Calcium Carbonate (Sturcal H) | 40.0 |
| Sodium Carrageenate (Viscarin) | 2.00 |
| Sodium Saccharin | 0.20 |
| Sodium Lauryl Sulphate (Empicol LZPV/C) | 1.50 |
| Flavour Composition | 1.00 |
| Total | 100.00 |

where Sturcal H, Viscarin and Empicol LZPV/C are all Trade Marks.

Any one of flavour compositions A-F above may be included in the following toothpaste, mouthwash, or chewing gum formulations, which axe prepared according to conventional methods known to those skilled in the art:

### Opacified Silica Toothpaste

| Material | % w/w |
|---|---|
| Sorbitol 70% syrup | 50.0 |
| Distilled Water | 23.6 |
| Sodium Monofluorophosphate | 0.80 |
| Trisodium Phosphate 12H₂O | 0.10 |
| Sodium Saccharin | 0.20 |
| Precipitated Silica (AC 30) | 8.00 |
| Precipitated Silica (TC 15) | 8.00 |
| Sodium Carboxy Methyl Cellulose (9M31XF) | 0.80 |
| Titanium Dioxide (Tiona) | 1.00 |
| Sodium Lauryl Sulphate (Empicol LZPV/C) | 1.50 |
| Polyethylene Glycol 1500 | 5.00 |
| Flavour Composition | 1.00 |
| Total | 100.00 |

Where Tiona and Empicol LZPV/C are Trade Marks.

### Ready-To-Use Mouthwash

### Mixture A—Alcohol Phase

| | % w/w |
|---|---|
| Ethanol 96%, Double Rectified | 12.000 |
| PEG 40 Hydrogenated Castor Oil (Cremophor RH40) | 0.250 |
| Flavour Composition | 0.200 |

### Mixture B—Aqueous Phase

| | % w/w |
|---|---|
| Sorbitol 70% syrup | 12.000 |
| Saccharin 25% solution | 0.200 |
| Cetyl Pyridinium Chloride | 0.025 |
| Distilled Water | 75.325 |

Where Cremophor RH40 is a Trade Mark.

The alcohol phase (mixture A) and aqueous phase (mixture B) were prepared separately and then combined to give the mouthwash.

### Chewing Gum

| Material | % w/w |
|---|---|
| Gum Base Balear T | 28.0 |
| Sorbitol Powder | 52.9 |
| Sorbitol Syrup | 6.0 |
| Xylitol | 6.0 |
| Glycerol 98% | 5.0 |
| Aspartame | 0.05 |
| Acesulfame K | 0.05 |
| Flavour Composition | 2.0 |

where Balear T and Acesulfame K are Trade Marks.

### EXAMPLE 5

### Malodour Counteraction Panel Test

Flavour compositions E and F embodying this invention were made and tested for their efficacy in reducing and/or preventing oral malodour in an Oral Malodour Panel Test.

The panel test is an objective test to assess and compare the effect on breath odour of oral care products containing a flavour composition against a control (a corresponding unflavoured oral care product).

The test is carried out double-blind so that neither judges, nor panellists know whether a control toothpaste (unflavoured toothpaste) or toothpaste containing a flavour composition in accordance with the invention is being tested. The panel test is carried out in a randomised, but balanced four- or five-week crossover experimental design, e.g. in a four week trial there are typically 3 test products containing a flavour composition and 1 control (unflavoured) product, so that each panellist tests every product on subsequent weeks. A “washout” toothpaste is used throughout each trial. That is, panellists are given a toothpaste of known flavour to use at home throughout the trial, which includes no antimicrobial actives e.g. triclosan. In this way, the efficacy of flavour compositions in accordance with the invention in terms of human-assessed oral malodour can be tested.

The effect on breath odour of the oral care products is assessed by two or three judges who have all undergone a full course of training in breath assessment. During the test, judges score panellists “blind” to other judges' scores, and these results are statistically evaluated to ensure that the judges' scores are comparable.

The panel is made up of 24 human subjects. Panellists are required to avoid spicy foods (e.g. garlic, onions) the night before each test day. They are also required to avoid all oral hygiene measures on the morning of a test, and not to consume coffee or to use perfumes or cosmetics. The test is carried out from 9 am until 1 pm.

During the test, a standard quantity (approximately 2 g) of a toothpaste: Opacified Silica Toothpaste of the formulation described in Example 4 above, containing one of the flavour compositions or an unflavoured control is applied to a toothbrush, and the panellist asked to brush their teeth in a normal fashion for one minute. Each panellist is allocated test or control products in accordance with a statistical design.

The breath is assessed organoleptically immediately prior to brushing (t=0) and then 20 minutes, 40 minutes and 60 minutes after brushing. The assessments are carried out on each panellist, who is firstly asked by one of the judges to close their mouth for a timed, two minute period. At the end of this two minute period, the panellist tilts their head back and opens their mouth, but without breathing. Each judge then sequentially sniffs mouth air from the panellist and organoleptically scores the breath odour on a standard scale of 0-5, where 0 is no odour and 5 is extreme malodour.

For each 20 minute period at which the breath is organoleptically assessed, the breath odour scored by a judge on the standard scale of 0-5 can be expressed as a percentage. In simplest terms, the breath malodour scores may be calculated as an approximate percentage as follows:

$$100 - ( \frac{( \begin{matrix}malodour score at t = 0 - \\ malodour score at time t\end{matrix} )}{malodour score at t = 0} \times 100 )$$

For example, if a panellist's organoleptic score at t=0 was 3, and this was reduced to 2 at t=60 minutes after brushing with a toothpaste containing, e.g. flavour composition E, then the malodour score would be 100−((3−2)/3×100)=66.6%. The malodour reduction would then be 33.33%. The malodour reduction is expressed as a percentage with reference to the baseline malodour score at t=0 of 100% and is calculated as follows, 100% (baseline at t=0)−malodour score (%).

In practice, the organoleptic scores are statistically analysed for each timed period i.e. immediately prior to brushing (t=0) and at 20, 40 etc minutes after brushing, using a general linear model in the computer statistics package SAS (PROC GLM) produced by SAS Institute Inc., SAS Campus Drive Cary, N.C. 27513-2414, USA, with fixed effects for judge, panellist, product, test session, and relevant interactions. The organoleptic scores are also analysed by ANOVA which provides an analysis of variance and yields mean values, based on the organoleptic scores of the 3 judges (corrected for inter-panellist variations) for each of the products in the trial, prior to product use, and at each timed period after product use. The percentage reductions can then be calculated, using the arithmetic means of the breath odour scores, either against the pre-treatment mean (time zero), or against the unflavoured control paste at the same time. In addition to the ANOVA statistics, model terms, means and confidence intervals are calculated for individual products. Post-hoc comparisons of pairs of products are assessed using student t-tests. For example, flavour compositions E and F at 60 minutes gave a 35% and 40%, respectively, malodour reduction compared with baseline (at pre-treatment/time zero).

FIGS. 1 and 2 show the results of the calculated malodour scores (%) against time (minutes) for toothpastes containing flavour composition E (FIG. 1) and flavour composition F (FIG. 2) compared with an unflavoured toothpaste as the control. It can be seen from the graphs that toothpastes incorporating flavour compositions E and F in-use give improved breath malodour reduction in human subjects compared with an unflavoured toothpaste.

### EXAMPLE 6

### Halimeter (HALIMETER is a Trade Mark) Device Assessed Breath Scores in Malodour Counteraction Panel Test

The effect on breath odour of toothpastes containing flavour compositions in accordance with the invention was investigated using the Halimeter™ device (Interscan Corp., PO Box 2496, Chatsworth, Calif., 91313-2496, USA).

Halimeter readings were taken in the following way.

Each panellist selected a fresh, “bendy” straw from a standard source. This was inserted securely into the silicone tubing of the Halimeter device. The panellist then avoided opening the mouth for two minutes. Following this period, the panellist inserted the end of the tube into their mouth, whilst biting gently on the ridged part of the straw, to ensure the insertion of the straw in the mouth to a standard depth.

A breath odour reading was taken on the Halimeter device prior to brushing. As in Example 5, when a panellist had brushed their teeth in a normal fashion for a one minute period with a control toothpaste or a toothpaste including a flavour composition, readings on the Halimeter device were recorded at 20 minutes, 40 minutes and 60 minutes after brushing.

1. A flavour composition which is a mixture of flavour materials, characterised in that the flavour composition comprises at least 8% by weight of the total weight of the flavour composition of ingredients selected from the following groups of flavour materials:
(a) at least 0.5% by weight of the flavour composition of one or more of the following: a peppermint oil comprising 1-isopropylidene-4-methyl-2-cyclohexanone in an amount from 1% to 4% by weight, 5-methyl-2-(1-methylethyl)-1-cyclohexanone in an amount from 8% to 13% by weight and less than 0.5% by weight of eucalyptol; a spearmint oil comprising less than 70% by weight of carvone and at least 14% by weight of limonene; or mixtures thereof; and
(b) at least 0.5% by weight of the flavour composition of two or more of the following: decanol, octanal, allyl hexanoate, anethole, anised rectified, basil oil, benzyl butyrate, camomile oil, cinnamic aldehyde, cis-3-hexenyl acetate, citral natural, citronella ceylon, ethyl heptanoate, eugenol, fennel sweet, geranyl acetate, ionone alpha, lime, orange flavour, para cresyl methyl ether, pinene alpha.

2. A flavour composition according to claim 1, wherein the peppermint oil and/or spearmint oil is of natural or synthetic origin, preferably of natural origin.

3. A flavour composition according to claim 1, wherein the peppermint oil comprises 1-isopropylidene-4-methyl-2-cyclohexanone in an amount from 1.5% to 3% by weight, 5-methyl-2-(1-methylethyl)-1-cyclohexanone in an amount from 8.5% to 12.5% by weight and less than 0.5% by weight of eucalyptol.

4. A flavour composition according to claim 2, wherein the peppermint oil is selected from one or more of the following: Peppermint Indian Rectified (all grades), Peppermint American Far West Bulked, Peppermint American Willamette Natural.

5. A flavour composition according to claim 2, wherein the spearmint oil is selected from one or more of the following: Spearmint American Far West Scotch, Spearmint Bulked Extra and Spearmint American Far West Native Redistilled.

6. A flavour composition according to claim 1, wherein the composition comprises at least 3, preferably at least 5 flavour materials from group (b).

7. A flavour composition according to claim 1, wherein the composition comprises at least 5% by weight, preferably at least 15% by weight, of the flavour composition of group (a) flavour materials.

8. A flavour composition according to claim 1, wherein the composition comprises at least 7% by weight, preferably at least 10% by weight, more preferably at least 20% by weight and even more preferably at least 30% by weight, of the flavour composition of group (b) flavour materials.

9. A flavour composition according to claim 1, wherein group (a) and group (b) flavour materials together comprise at least 20% by weight of the total weight of the flavour composition, preferably at least 40% by weight.

10. A flavour composition according to claim 1, wherein the composition additionally includes one or more of the following materials: tea tree oil, aldehyde C9, Orange Oil Terpeneless and aldehyde C10.

11. A consumer product comprising a flavour composition according to claim 1.

12. A consumer product according to claim 11, wherein the consumer product is an oral care product.

13. A method of reducing and/or preventing oral malodour comprising administering a flavour composition comprising one or more of the following flavour materials: octanal, allyl hexanoate, anethole, aniseed rectified, basil oil, benzyl butyrate, camomile oil, cinnamic aldehyde, cis-3-hexenyl acetate, ethyl heptanoate, fennel sweet, ionone alpha, lime, orange flavour, para cresyl methyl ether, pinene alpha, a spearmint oil comprising less than 70% by weight of carvone and at least 14% by weight of limonene, a peppermint oil comprising 1-isopropylidene-4-methyl-2-cyclohexanone in an amount from 1% to 4% by weight, 5-methyl-2-(1-methylethyl)-1-cyclohexanone in an amount from 8% to 13% by weight and less than 0.5% by weight of eucalyptol.

14. A method for reducing or preventing oral malodour by introducing in the oral cavity a flavour composition, in accordance with claim 1.

15. A method for reducing or preventing the production of odoriferous volatile sulphur compounds in the oral cavity, the method comprising the step of introducing in the oral cavity a flavour composition, in accordance with claim 1.

16. A method for inhibiting the bacterial production in vitro of odoriferous volatile sulphur compounds, by introducing a flavour composition in accordance with claim 1 to a bacterial culture.

17. A method for reducing or preventing oral malodour by introducing in the oral cavity a consumer product in accordance with claim 11.
